(19) United States
(12) Patent Application Publication
(10) Pub. No.: US 2021/0186934 A1
(43) Pub. Date: Jun. 24, 2021

(54) COMPOSITIONS AND USES THEREOF

(71) Applicant: LIVERPOOL JOHN MOORES UNIVERSITY, Liverpool (GB)

(72) Inventors: Oladimeji Kehinde ROSS, Liverpool (GB); Adam RICHARDSON, Liverpool (GB)

(73) Assignee: LIVERPOOL JOHN MOORES UNIVERSITY, Liverpool (GB)

(21) Appl. No.: 17/057,333

(22) PCT Filed: May 21, 2019
(86) PCT No.: PCT/GB2019/051402

§ 371 Date: Nov. 20, 2020

(30) Foreign Application Priority Data
May 21, 2018 (GB) 1808298.2
May 31, 2018 (GB) 1808907.8

(51) Int. Cl. A61K 31/433; A61K 33/06; A61K 45/06; A61P 17/02; C12N 15/113; A61K 47/54

(52) U.S. Cl. CPC A61K 31/433; A61K 33/06; A61K 45/06; C12N 2310/351; C12N 15/113; A61K 47/549; C12N 2310/141; A61P 17/02

(57) ABSTRACT

The present invention relates to compositions comprising a miR-184 modulator, a miR-184, or mimetic, or derivative thereof, an expression vector encoding miR-184, or a carrier molecule conjugated to miR-184 for regulating keratinocyte migration and/or differentiation. The invention has particular use as a medicament for the prevention, management, amelioration or treatment of damaged or defective keratinocytes in a skin condition or disease.

### TECHNICAL FIELD OF THE INVENTION

The present invention relates to compositions comprising miR-184 modulators for regulating keratinocyte migration and/or differentiation and has particular use in, but not limited to, the prevention, management, amelioration or treatment of damaged or defective keratinocytes in a skin condition or disease.

### BACKGROUND TO THE INVENTION

The epidermis is a stratified tissue maintained by controlled proliferation and terminal differentiation of keratinocytes (Eckhart et al., 2013). Extracellular calcium (Ca²⁺) induces involucrin (IVL) and several other differentiation proteins to enable formation of the cornified envelope. Differentiating suprabasal keratinocytes express cell cycle proteins such as cyclin A, B and E to support their enlargement (Zanet et al., 2010). Furthermore, cyclin E, which drives the G1/S phase transition, accumulates within IVL-expressing suprabasal layers and promotes the proliferation:differentiation switch through mitotic failure and DNA damage (Freije et al., 2012, Zanet et al., 2010).

MicroRNAs (miRNAs) are short noncoding RNAs (18-25 nucleotides) that attenuate post-transcriptional gene output through translational inhibition and destabilization of mRNA transcripts, the latter appearing to sustain the bulk of steady-state repression (Eichhorn et al., 2014, Huntzinger and Izaurralde, 2011). Several miRNAs have been implicated in epidermal differentiation including miR-203, miR-205 and miR-24 (reviewed (Riemondy et al., 2014)). Furthermore, over 50 miRNAs were upregulated in human keratinocytes exposed to high extracellular Ca²⁺ (Hildebrand et al., 2011) and miR-24 was shown to be upregulated by Ca²⁺-during keratinocyte differentiation (Amelio et al., 2012). Given that store-operated Ca²⁺ entry (SOCE) through the STIM1: ORAI1 axis plays essential roles in keratinocyte and epidermal physiology (Numaga-Tomita and Putney, 2013, Ross et al., 2007, Vandenberghe et al., 2013), it is conceivable that Ca²⁺-dependent induction of miRNAs relies at least partly on SOCE. However, the impact of SOCE on miRNA expression has received little attention.

Very recent studies detected miR-184 expression predominantly in the spinous layer of neonatal mouse epidermis, thus implicating miR-184 in epidermal differentiation (Nagosa et al., 2017). Modulation of miR-184 levels in mouse skin and cultured human keratinocytes together revealed that miR-184 represses keratinocyte proliferation and supports commitment to differentiation through the Notch axis. Although not listed among miRNAs upregulated by high Ca²⁺ in earlier work (Hildebrand et al., 2011), a 4-fold increase in miR-184 expression was observed in human keratinocytes exposed to high Ca²⁺ for 7 days (Nagosa et al., 2017). However, the mechanisms of miR-184 induction have not been defined and little is known about the signalling pathways regulating miR-184 expression.

The inventors had previously observed expression of miR-184 in reconstituted human epidermis (RHE) and in the HaCaT keratinocyte cell line (Roberts et al., 2013). In contrast, Lavker and colleagues did not detect miR-184 in monolayer cultures of proliferating epidermal keratinocytes (Yu et al., 2008). Exposure of RHE to inflammatory cytokines interleukin-22 (IL-22) or oncostatin M (OSM) enhanced miR-184 expression in studies, suggesting that miR-184 levels can be modulated by external signals (Roberts et al., 2013).

Despite significant advances in understanding the multiple factors associated with chronic wounds, there remains a significant unmet need for therapeutic interventions to promote wound healing. With the ageing population and associated rise in the incidence of diabetes, pressure ulcers, venous leg ulcers and diabetic foot ulcers, the clinical and socioeconomic challenges presented by non-healing wounds are likely to persist, exerting enormous pressure on health services in both industrialised and developing nations (Eming et al., 2014, Nunan et al., 2014, Whittam et al., 2016). Estimates put the costs of managing wounds and associated comorbidities at £5.3 billion annually in the UK (Guest et al., 2015) and a staggering $25 billion in the USA (Sen et al., 2009).

The formation of new epidermal tissue over the denuded wound surface is fundamental to the completion of wound healing. Such re-epithelialization is mediated by keratinocyte migration from the wound edge to repopulate the exposed extracellular matrix, a processes that is impaired in chronic wounds (Usui et al., 2008). Emerging evidence shows that microRNA (miRNAs) modulate keratinocyte migration in order to regulate re-epithelization, as reviewed recently (Ross, 2018): miR-21, miR-31 and miR-132 promote keratinocyte migration (Li et al., 2015, Li et al., 2017, Wang et al., 2012, Yang et al., 2011). In contrast, over expression of miR-24 or miR-483-3p inhibited HPEK migration (Amelio et al., 2012, Bertero et al., 2011). Keratinocyte migration is also regulated by miR-205, but both enhancement and inhibition of miR-205 have been linked to re-epithelization (Wang et al., 2016, Yu et al., 2010).

It is an object of the present invention to overcome or alleviate one of more of the above identified problems. It is also an object of the present invention to provide treatments for a number of skin disorders and conditions. It would be preferable if such a treatment could also be used in wound healing.

### SUMMARY OF THE INVENTION

In accordance with a first aspect of the present invention, there is provided a composition comprising a miR-184 modulator for regulating keratinocyte migration and/or differentiation.

The miR-184 modulator may be adapted to increase expression or prevalence of miR-184 so as to promote keratinocyte migration and/or differentiation. The promotion of keratinocyte migration and/or differentiation may be in damaged or defective keratinocytes and the promotion of keratinocyte differentiation may be for re-epithelialization.

The composition may be for use as a medicament.

In a related aspect, there is a provided a composition for use in the prevention, management, amelioration or treatment of damaged or defective keratinocytes in a skin condition or disease, the composition comprising a miR-184 modulator.

In another related aspect, there is provided a method of prevention, management, amelioration or treatment of damaged or defective keratinocytes in a skin condition or disease, the method comprising administering a therapeutically effective amount of the composition to a subject in need thereof, the composition comprising a miR-184 modulator.

In a related aspect, the invention may comprise the composition, for use in the manufacture of a medicament for the prevention, management, amelioration or treatment of damaged or defective keratinocytes in a skin condition or disease, the composition comprising a miR-184 modulator.

The skin condition may comprise a wound, including chronic wounds.

The composition as claimed in any preceding claim, where the composition further comprises bioavailable calcium.

The composition may further comprise a store-operated calcium entry modulator. The store-operated calcium entry modulator may comprise a calcium release-activated channel (CRAC) inhibitor. The calcium release-activated channel (CRAC) inhibitor may comprise a number of inhibitors, such as BTP2 and/or derivatives thereof.

In accordance with a second aspect of the present invention, there is provided a composition comprising miR-184, or mimetic, or derivative thereof, for the promotion of keratinocyte migration and/or differentiation.

The promotion of keratinocyte migration and/or differentiation may be in damaged or defective keratinocytes and may be for re-epithelialization.

In a different embodiment, the miR-184 modulator may be for decreasing expression or prevalence of miR-184 so as to prevent or reduce keratinocyte migration and/or differentiation. The prevention or reduction of keratinocyte migration and/or differentiation and may be used for the prevention or reduction of re-epithelialization.

The composition may be for use as a medicament.

In a related aspect, there is provided a composition for use in the prevention, management, amelioration or treatment of damaged or defective keratinocytes in a skin condition, the composition comprising miR-184, or mimetic, or derivative thereof.

In another related aspect, there is provided a method of prevention, management, amelioration or treatment of damaged or defective keratinocytes in a skin condition or disease, the method comprising administering a therapeutically effective amount of the composition to a subject in need thereof, the composition comprising miR-184, or mimetic, or derivative thereof.

In a related aspect, the invention may comprise a composition, for use in the manufacture of a medicament for the prevention, management, amelioration or treatment of damaged or defective keratinocytes in a skin condition or disease, the composition comprising miR-184, or mimetic, or derivative thereof.

The skin condition may comprise a wound, including chronic wounds.

The composition as claimed in any preceding claim, where the composition further comprises bioavailable calcium.

The composition may further comprise a store-operated calcium entry modulator. The store-operated calcium entry modulator may comprise a calcium release-activated channel (CRAC) inhibitor. The calcium release-activated channel (CRAC) inhibitor may comprise a number of inhibitors, such as BTP2 and/or derivatives thereof.

In accordance with a third aspect of the present invention, there is provided a composition comprising an expression vector encoding miR-184, or a carrier molecule conjugated to miR-184, for the promotion of keratinocyte migration and/or differentiation.

The promotion of keratinocyte migration and/or differentiation may be in damaged or defective keratinocytes and the promotion of keratinocyte differentiation may be for re-epithelialization.

The composition may be for use as a medicament.

In accordance with a related aspect, there is provided a composition is for use in the prevention, management, amelioration or treatment of damaged or defective keratinocytes in a skin condition, the composition comprising an expression vector encoding miR-184, or a carrier molecule conjugated to miR-184.

In another related aspect, there is provided a method of prevention, management, amelioration or treatment of damaged or defective keratinocytes in a skin condition or disease, the method comprising administering a therapeutically effective amount of the composition to a subject in need thereof, the composition comprising an expression vector encoding miR-184, or a carrier molecule conjugated to miR-184.

In a related aspect, the invention may comprise a composition, for use in the manufacture of a medicament for the prevention, management, amelioration or treatment of damaged or defective keratinocytes in a skin condition or disease, the composition comprising an expression vector encoding miR-184, or a carrier molecule conjugated to miR-184.

The skin condition may comprise a wound, including chronic wounds.

The composition as claimed in any preceding claim, where the composition further comprises bioavailable calcium.

The composition may further comprise a store-operated calcium entry modulator. The store-operated calcium entry modulator may comprise a calcium release-activated channel (CRAC) inhibitor. The calcium release-activated channel (CRAC) inhibitor may comprise a number of inhibitors, such as BTP2 and/or derivatives thereof.

If the composition comprises an expression vector, preferably, it is provided as a gene therapy vector. The vector may be viral or non-viral (e.g. a plasmid). Viral vectors include those derived from adenovirus, adeno-associated virus (AAV) including mutated forms, retrovirus, lentivirus, herpes virus, vaccinia virus, MMLV, GaLV, Simian Immune Deficiency Virus (SIV), HIV, pox virus, and SV40. A viral vector is preferably replication defective, although it is envisaged that it may be replication deficient, replication competent or conditional. A viral vector may typically persist in an extrachromosomal state without integrating into the genome of the target neural cells. A preferred viral vector is an AAV vector. Selective targeting may be achieved using a specific AAV serotype (AAV serotype 2 to AAV serotype 12) or a modified version of any of these serotypes including true type variants.

The viral vector may be modified to delete any non-essential sequences. For wild type AAV, replication is unable to take place without the presence of helper virus, such as adenovirus. For recombinant adeno-associated virus, preferably the replication and capsid genes are provided in trans (in pRep/Cap plasmid), and only the 2 ITRs of AAV genome are left and packaged into a virion, while the adenovirus genes required are provided either provided by adenovirus or another plasmid. Similar functional modifications may be made to a lentiviral vector where appropriate.

The viral vector will preferably have the ability to enter a cell. However, a non-viral vector such as plasmid may be complexed with an agent to facilitate its uptake by a target cell. Such agents include polycationic agents. Alternatively, a delivery system such as a liposome based delivery system may be used.

The inventors have unexpectedly and advantageously found that exogenous Ca²⁺ stimulates miR-184 expression in primary epidermal keratinocytes and that this occurs in a SOCE-dependent manner. Levels of miR-184 were found to be raised by about 30-fold after exposure to 1.5 mM for 5 days. In contrast, neither phorbol ester nor 1, 25-dihydroxyvitamin D₃ had any effect on miR-184 levels. Pharmacologic and genetic inhibitors of SOCE abrogated Ca²⁺-dependent miR-184 induction by 70% or more. Ectopic miR-184 slow keratinocyte proliferation and led to a 4-fold increase in the expression of involucrin, a marker of early keratinocyte differentiation. Exogenous miR-184 also triggered a 3-fold rise in levels of cyclin E and doubled the levels of γH2AX, a marker of DNA damage. The p21 cyclin-dependent kinase (CDK) inhibitor, which supports keratinocyte growth arrest, was also induced by miR-184. Together these findings point to a SOCE:miR-184 pathway that targets a cyclin E/DNA damage regulatory node to facilitate keratinocyte differentiation.

Furthermore, it was identified that ectopic miR-184 itself promoted HPEK differentiation and this was associated with the elevation of cyclin E, DNA damage and induction of p21. Exogenous miR-184 also triggered a 3-fold rise in levels of cyclin E and doubled the levels of γH2AX, a marker of DNA damage. The p21 cyclin-dependent kinase (CDK) inhibitor, which supports keratinocyte growth arrest, was also induced by miR-184.

As used herein, the terms “treatment”, “treating”, “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting or slowing its development; and (c) relieving the disease, i.e., causing regression of the disease.

The term “subject” used herein includes any human or nonhuman animal. The term “nonhuman animal” includes all mammals, such as nonhuman primates, sheep, dogs, cats, cows, horses.

A “therapeutically effective amount” refers to the amount of an active ingredient (such as miR-184 or miR-184 mimetic) that, when administered to a subject for treating a disease, is sufficient to affect such treatment for the disease. The “therapeutically effective amount” will vary depending on active ingredient(s) used, the disease and its severity and the age, weight, etc., of the subject to be treated.

The compositions may suitably be in the form of a liquid, solution (e.g., aqueous, non-aqueous), suspension (e.g., aqueous, non-aqueous), emulsion (e.g., oil-in-water, water-in-oil), elixir, syrup, electuary, mouthwash, cavity wash, drops, granules, powders, ampoule, bolus, suppository, pessary, tincture, gel, paste, ointment, cream, lotion, oil, foam, spray, mist, or aerosol.

The compositions may suitably be provided as part of a patch, adhesive plaster, bandage, dressing, or the like which is impregnated with one or more active compounds and optionally one or more other pharmaceutically acceptable ingredients, including, for example, penetration, permeation, and absorption enhancers. The composition may also suitably be provided in the form of a depot or reservoir.

The compositions according to the aspects of the invention may further comprise one or more pharmaceutically or cosmetically acceptable ingredients or excipients. Pharmaceutically acceptable ingredients are well known to those skilled in the art, and include, but are not limited to, pharmaceutically acceptable carriers, adjuvants, excipients, diluents, fillers, buffers, preservatives, 3 carriers, excipients, diluents, fillers, buffers, preservatives, anti-oxidants, lubricants, stabilisers, solubilisers, surfactants (e.g. wetting agents), masking agents, colouring agents, fragrance agents and penetration agents.

Preferably the composition is formulated for topical administration particularly for use or application to, or on, the skin.

The compositions may be formulated for topical administration in the form of gels, pastes, ointments, creams, lotions, and oils, as well as patches, adhesive plasters, bandages, dressings, depots, cements, glues, and reservoirs. Preferably the composition may be formulated for topical administration in the form of a cream, gel, ointment or oil.

Ointments are typically prepared from the composition and a paraffinic or a water-miscible ointment base.

Creams are typically prepared from an active ingredient and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

Emulsions may be formed with the active ingredient and such emulsions may be formed by using a suitable emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulsion and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

The compositions may be administered alone or in combination with other treatments, either simultaneously or sequentially. Compositions according to the invention may further comprise other active agents, for example anti-bacterial agents such as bactericidal agents.

In some embodiments, the compositions may be provided as a suspension in a pharmaceutically or cosmetically acceptable excipient, diluent or carrier.

The compositions of the present invention may be formulated as medicaments, that is to say formulated as a medicine, or a medical device. The medicament may include other pharmaceutically acceptable ingredients well known to those skilled in the art, including, but not limited to, pharmaceutically acceptable carriers, adjuvants, excipients, diluents, fillers, buffers, preservatives, anti-oxidants, lubricants, stabilisers, solubilisers, surfactants (e.g. wetting agents), masking agents, colouring agents, flavouring agents, and sweetening agents. The formulation may further comprise other active agents, for example other therapeutic or prophylactic agents.

Features, integers, characteristics, compounds, molecules, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and figures), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### DETAILED DESCRIPTION OF THE INVENTION

Embodiments of the invention are described below, by way of example only, with reference to the accompanying figures in which:

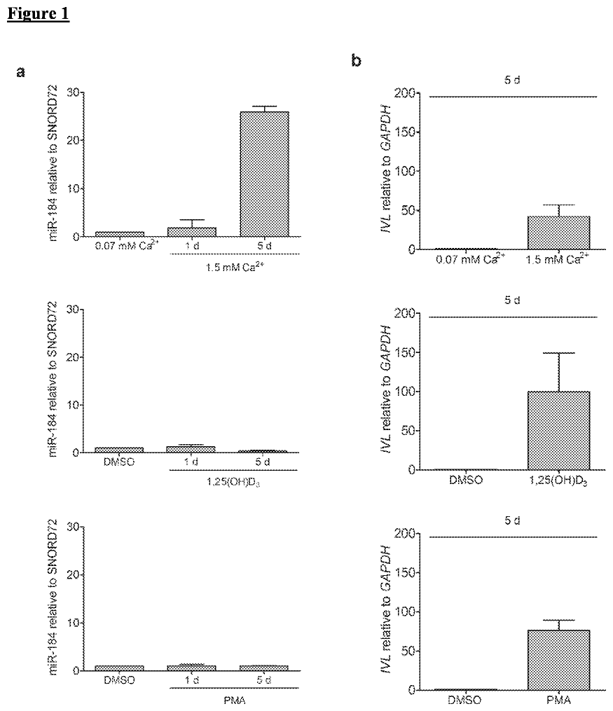

FIG. 1: Induction of miR-184 during Ca²⁺-dependent HPEK differentiation. (a) HPEKs grown to confluence were treated with high (1.5 mM) Ca²⁺, 100 nM 1,25(OH)D₃, 100 nM PMA or dimethyl sulfoxide (DMSO) vehicle for 1 or 5 days (d). (b) Upregulation of the HPEK differentiation marker involucrin (IVL) during 5 d exposure to 1.5 mM Ca²⁺, 100 nM 1,25(OH)D₃ or 100 nM PMA. Data shown represent means +SEM from 3 independent experiments. Expression was normalized to SNORD72 for miR-184 and GAPDH for IVL. Values are presented relative to 0.07 mM Ca²⁺ or DMSO-treated controls;

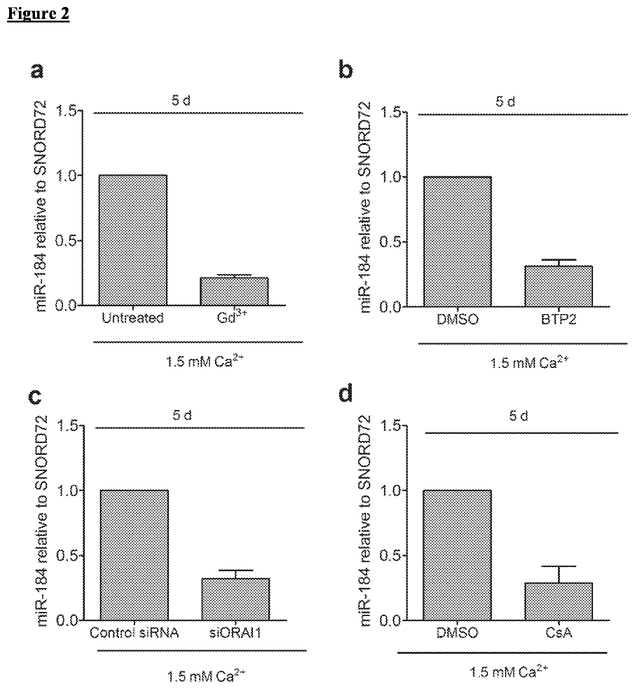

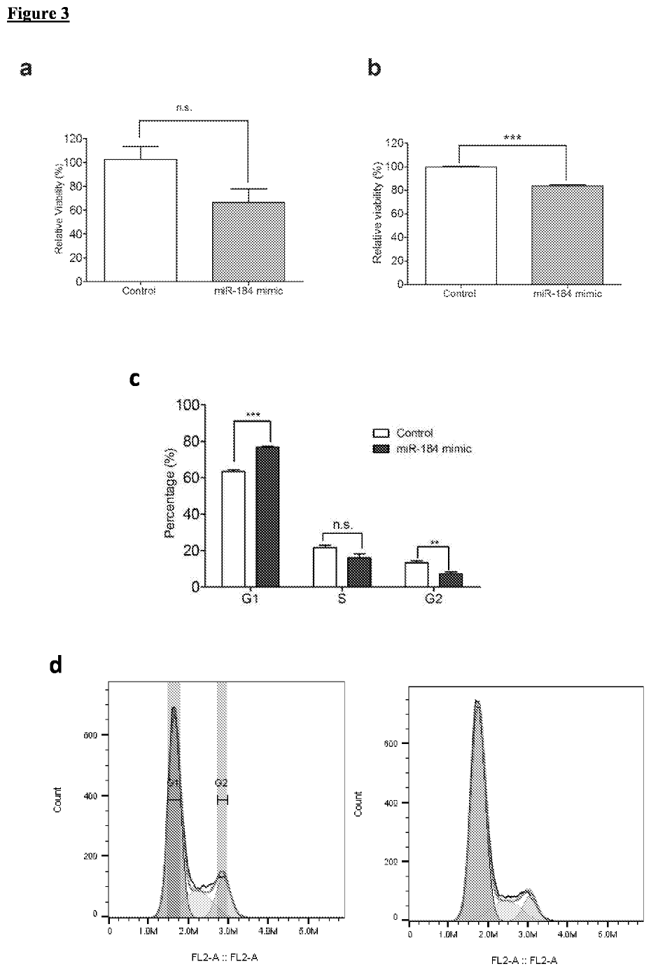

FIG. 2: Inhibition of SOCE attenuates Ca²⁺-dependent miR-184 expression. HPEKs were maintained in 1.5 mM Ca²⁺ for 5 d with or without (a) 1 μM Gd³⁺, (b) 1 μM BTP2, (c) 100 nM ORAI1-targeting siRNA or (d) 1 μM CsA as indicated. The Gd³⁺, BTP2 and CsA were added 1 h prior to Ca²⁺ switch and refreshed after day 2. Data shown represent means +SEM from 3 independent experiments. Expression was normalized to SNORD72 for miR-184 and GAPDH for IVL. Values are presented relative to untreated, DMSO-treated or control siRNA as indicated;

FIG. 3: miR-184 reduces HPEK proliferation. HPEK were nucleofected with 100 nM of the miR-184 mimic, miR-184 inhibitor or control oligonucleotide as indicated. The effect of miR-184 on HPEK viability determined with MTT (a) or trypan blue staining (b). Cell cycle profiles were assessed using propidium iodide staining and flow cytometry (c). Representative histograms from HPEK loaded with miR-184 mimic (left) or control oligonucleotide (right) are presented in (d). ***, p<0.001; **, p<0.01; n.s., not significant;

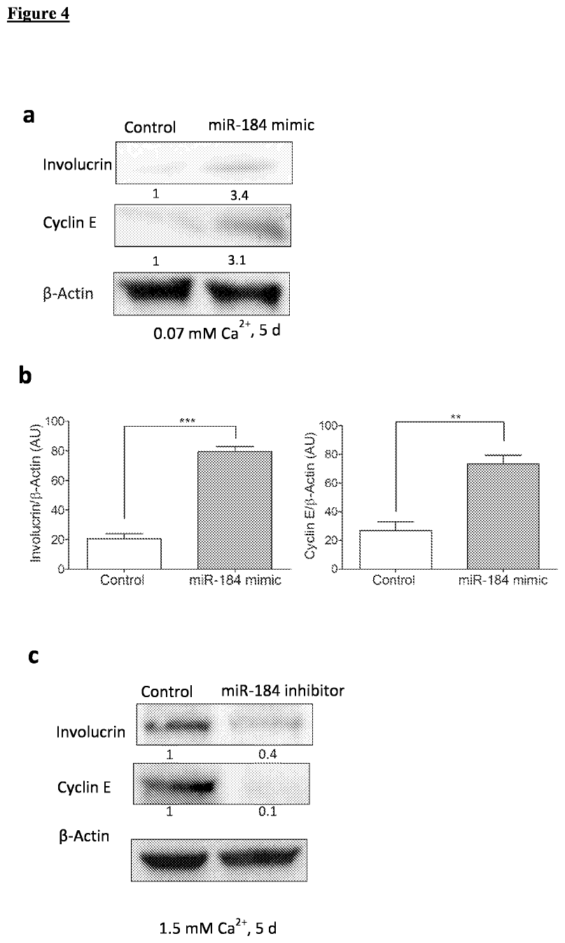

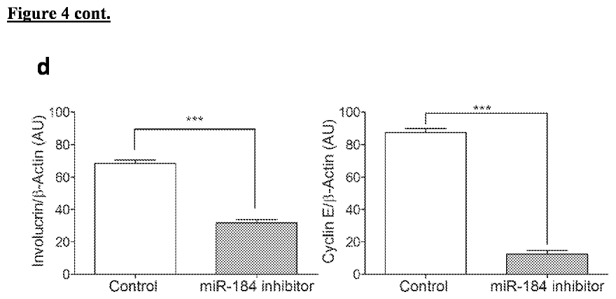

FIG. 4: miR-184 induces IVL and cyclin E expression in HPEK. Cells loaded with 100 nM miR-184 mimic or a control oligonucleotide were maintained in low Ca²⁺ (0.07 mM) media (a,b) or high Ca²⁺ (1.5 mM) media (c,d) for 5 d prior to western blotting. Graphs (b,d) show the mean+SEM densitometry levels relative to β-actin. Data shown were pooled from 3 independent experiments. ***, p<0.001; **, p<0.01;

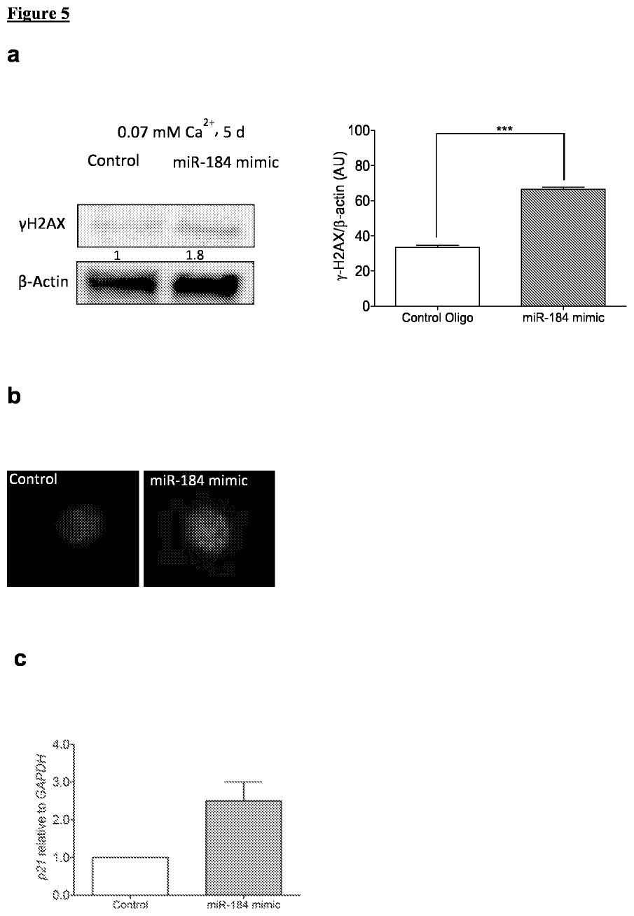

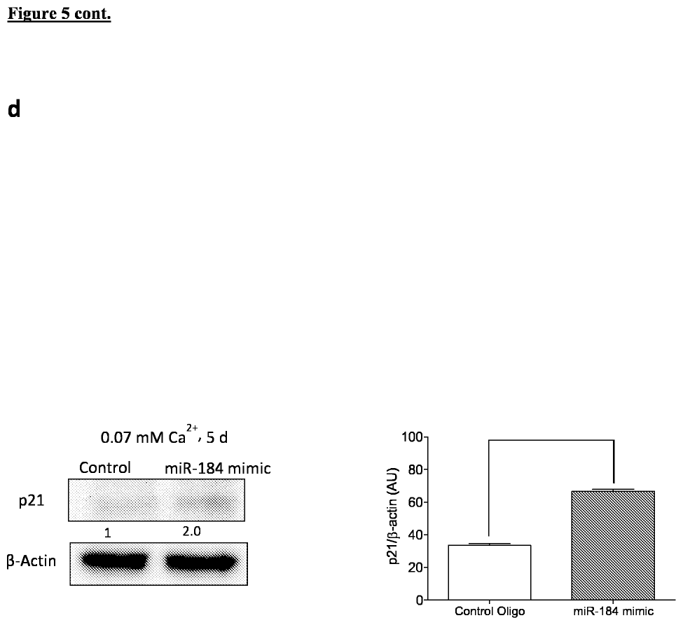

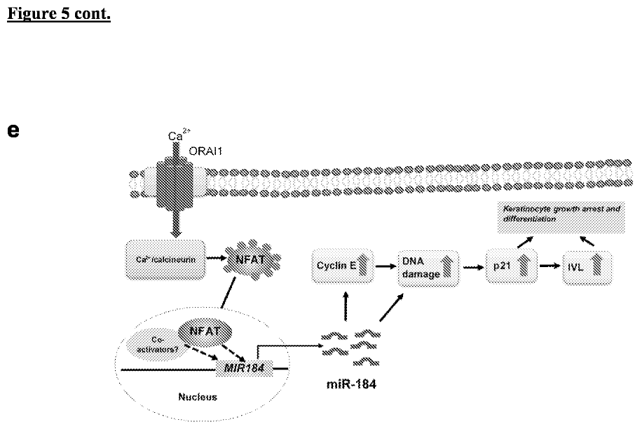

FIG. 5: miR-184 promotes DNA damage and p21 expression in HPEK. Cells loaded with 100 nM miR-184 mimic or a control oligonucleotide were maintained in low Ca²⁺ (0.07 mM) media for 5 d prior to western blotting for γH2AX (a). Immunofluorescent staining for γH2AX in cells loaded with 100 nM miR-184 for 5 d, with quantification of foci number and intensity (b). Elevated expression of p21 at the transcript (c) and protein (d) levels in HPEK loaded with 100 nM miR-184 for 5 d. Graphs in (a) and (d) show the mean+SEM densitometry levels relative to β-actin, pooled from three independent experiments. ***, p<0.0001. Schematic representation of the SOCE:miR-184 axis to keratinocyte differentiation;

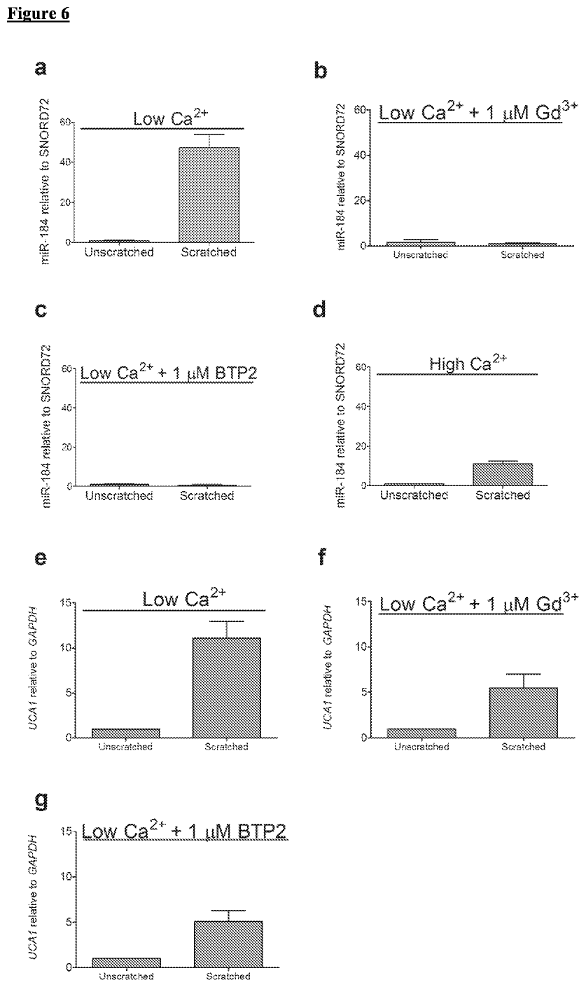

FIG. 6: Induction of miR-184 in scratched monolayers. Confluent HPEK monolayers were scratched and cells harvested after 5 days in low (0.07 mM) or high (1.5 mM) Ca²⁺ media as indicated. The SOCE blockers Gd³⁺ and BTP2 and were added 1 h after scratching and refreshed after day 2. (a-d) miR-184 expression normalized to SNORD72. (c-d) LncRNA UCA1 expression normalised to GAPDH; and

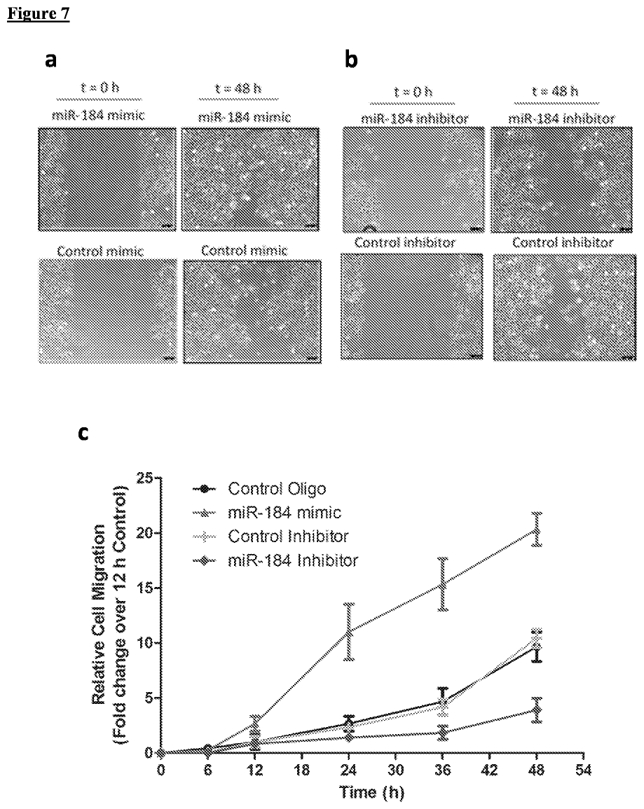

FIG. 7: miR-184 promotes keratinocyte migration. Cells were loaded with 100 nM of miR-184 mimic (a) or miR-184 inhibitor (b) or respective control oligonucleotides. Monolayers were scratched upon reaching confluence and monitored for 48 h. (c) Migration rates were normalised to the number of cells populating the breach at 12 h. Data pooled from 3 independent experiments.

### EXAMPLE 1—MICRORNA-184 INDUCTION BY STORE-OPERATED CALCIUM ENTRY AND REGULATION OF EARLY KERATINOCYTE DIFFERENTIATION

Experiments were conducted to establish whether MicroRNA-184 would be induced by Store-Operated Calcium Entry (SOCE).

### Reagents

Oligonucleotides (miR-184 mimic, siORAI and respective controls) were purchased from GE Healthcare (Little Chalfont, UK). The locked nucleic acid (LNA) miR-184 inhibitor and a non-targeting control were from Exiqon (Vedbaek, Denmark). Gadolinium(III) chloride (Gd³⁺) and differentiation reagents (1, 25-(OH)₂D3/calcitriol and PMA) and were purchased from Bio-Techne (Abingdon, UK). BTP2 (also known as YM58483) was purchased from Abcam (Cambridge, UK).

### Keratinocyte Isolation, Culture and Differentiation

Human progenitor epidermal keratinocytes (HPEK) were isolated from human foreskin or purchased from CellnTec (Bern, Switzerland). For isolation, tissues were washed with phosphate-buffered saline, trimmed of any subcutaneous fat, connective tissues and blood vessels before digestion with 20 mg/ml dispase (Sigma) at 4° C. overnight with further digestion for 1 hour at room temperature. Thereafter, the epidermis was separated and incubated in 0.5% TrpLE (Thermofisher scientific, Cheshire, UK) for 5 min at 37° C., 5% CO₂. Keratinocytes were centrifuged (1200 rpm for 5 min) and re-suspended in CnT-Prime (CellnTec) supplemented with 1% penicillin/streptomycin/amphotericin B (PSA) and IsoBoost supplement CnT-ISO (CellnTec) to enhance isolation efficiency. Culture medium was changed every 2-3 days until cells reached 80% confluence with PSA exclusion from the culture medium after the first passage. Keratinocytes were sub-cultured using CnT-Accutase (CellnTec) and re-cultured at 4×10³ cells per cm². For differentiation, keratinocytes were at 3×10⁵/well of a 6-well plate in CnT-Prime and allowed to reach confluence before adjusting the medium to 1.5 mM calcium using CaCl₂.

### Oligonucleotide Nucleofection

Keratinocytes were sub-cultured and 5×10⁵ cells resuspended in 100 μl nucleofection solution from the P3 Primary Cell 4D-Nucleofector kit (Lonza, Castleford, UK) and with 100 nM miRNA human miR-184 mimic (a synthetic double-stranded oligonucleotide mimicking endogenous miR-184) or non-targeting negative control oligonucleotide. Cell suspensions were then transferred to nucleofection cuvettes and pulsed on the DS-138 programme of a 4D-Nucleofector. After incubation in pre-equilibrated CnT-Prime at room temperature for 10 min, the nucleofected cells were transferred to multiwall plates and incubated at 37° C., 5% CO₂ incubator with the media replacement the following day.

### Cell Viability

Cells transfected with the miR-184 mimic or negative control were seeded at 2×10⁴/well of a 96-well plate and maintained in CnT-Prime for 3 d. The MTT reagent 3-(4,5-Dimethylthiazol-2-yl)-2,5-diphenyltetrazolium bromide was added to each well at 5 mg/ml and incubated at 37° C., 5% CO₂ for 4 h. Culture medium was removed, the 96-well plate air dried and 100 μl dimethyl sulfoxide (DMSO) added to each well. After shaking for 5 min to ensure solubilisation of formazan crystals, the absorbance of each well was read on a Clariostar plate reader (BMG Labtech, Aylesbury, UK) at OD 470 nm. All experiments were performed in triplicate and at least three times. For trypan blue viability tests, an aliquot of cells nucleofected with miR-184 mimic or negative control oligo was mixed 1:1 with 0.4% trypan blue before loading onto a haemocytometer. Dark blue cells were counted as non-viable cells and those with bright centres counted as live.

### Cell Cycle

Nucleofected cells seeded at 3×10⁵/well of a 6-well plate were grown for 2 d, detached using CnT-Accutase (CellnTec, Bern, Switzerland) washed twice with PBS then fixed in 70% ethanol for 24-72 h. Propidium iodide (100 μg/ml) was added for 30 min in the dark at room temperature then cells were analysed using a BD Accuri C6 flow cytometer (BD Biosciences, Wokingham, UK) and FlowJo version 10.0 software.

Semi-quantitative Reverse Transcriptase PCR (sqRT-PCR)

Total RNA was isolated from cells using the AllPrep DNA/RNA/miRNA Universal kit (Qiagen, Manchester, UK). RNA concentration was determined using a NanoDrop™ 2000c. Complementary DNA (cDNA) was synthesised from 400 ng of RNA using the miScript II RT kit (Qiagen) with HiFlex buffer. PCR amplifications were performed with Quantifast SYBR Green and QuantiTect miRNA/universal primers or RT² mRNA primer assays, all from Qiagen. Thermocycling was performed on a Rotor-Gene® as follows: 95° C. for 15 min, followed by 40 cycles of 94° C. for 15 s, 55° C. for 30 s and 70° C. for 30 s. Relative expression of miRNA and mRNA determined using the 2^{-ΔΔCT} relative quantification method (Livak and Schmittgen, 2001).

### Western Blotting

Cells were lysed in RIPA buffer containing protease and phosphatase inhibitors and 20 or 40 μg of total protein resolved on mini-PROTEAN 12% SDS/PAGE precast gels (Bio-Rad, Watford, UK). After transfer to polyvinylidene difluoride (PVDF) membranes, samples were incubated with primary antibodies: Involucrin (1:1000; Novus biologicals, Oxon, UK), Cyclin E (1:750; bio-Techne, Abingdon, UK), γH2AX (1:500; Millipore, Watford, UK, p21 (1:500; Bio-Rad, Oxfordshire, UK), GAPDH (1:5000, R&D systems) and β-actin (1:2000; Sigma) overnight at 4° C. Membranes were then washed several times with Tris-buffered saline with 0.1% tween-20 (TBST) and incubated with horseradish peroxidase (HRP) conjugated secondary antibodies for 1 hour at room temperature. Membranes were washed before chemiluminescence was visualised using Clarity ECL reagents (Bio-Rad). ImageJ software was used to perform densitometry with target protein values normalised to the corresponding β-actin controls.

### Statistics

Where indicated, the Students' t test was performed using Graph Pad Prism 5.0 (La Jolla, Calif., USA).

Induction of miR-184 in Human Primary Epidermal Keratinocytes

Expression of miR-184 in HPEK was evaluated and maintained in parallel cultures under low (0.07 mM) to sustain proliferation or high (1.5 mM) extracellular Ca²⁺ to promote differentiation. As shown in FIG. 1A, relative miR-184 expression was almost 30-fold higher in HPEK treated with 1.5 mM Ca²⁺ for 5 days compared to cells maintained in low Ca²⁺. Only very low levels of miR-184 were detected in the cells under low Ca²⁺ conditions, suggesting the high Ca²⁺ challenge triggers de novo miR-184 expression. In contrast, the active form of vitamin D, 1, 25-dihydroxyvitamin D₃, (1, 25-(OH)₂D3; Calcitriol) or phorbol 12-myristate 13-acetate (PMA) did not promote miR-184 expression in HPEK over the time points assessed (FIG. 1a). By comparison, both these agents evoked robust IVL expression as expected (FIG. 1b) given their established functions as inducers of keratinocyte differentiation (Bikle, 2004, Karlsson et al., 2010). Together, these observations suggest that the induction of miR-184 by Ca²⁺ is associated with Ca²⁺-dependent pathways and not simply keratinocyte differentiation. Interferon gamma (IFNγ) has also been reported to trigger keratinocyte differentiation (Karlsson et al., 2010) but no induction of miR-184 was observed with 10 ng/ml IFNγ, and the low levels of miR-184 registered in untreated cells became undetectable following IFNγ stimulation for 1 or 5 days (data not shown).

To determine whether Ca²⁺-dependent induction of miR-184 was associated with SOCE, HPEK were maintained in high Ca²⁺ for 5 days in the presence of the SOCE blocker gadolinium (III) Gd³⁺ or the pharmacologic ORAI1 inhibitor BTP2. Alternatively, ORAI1 expression was ablated using short-interfering RNA (siRNA) that was transferred into the cells using nucleofection 1 d prior to Ca²⁺ elevation. As shown in FIG. 2, Gd³⁺, BTP2 and siORAI1 reduced Ca²⁺-induced miR-184 expression by 70% or more. The siRNA suppressed ORAI1 levels by about 55%, whereas Gd³⁺ and BTP2 had little effect on ORAI1 expression (data not shown). Together, these findings suggest elevation of miR-184 during Ca²⁺-dependent keratinocyte differentiation is mediated at least partly by Ca²⁺ influx through ORAI1.

The Ca²⁺⁻calmodulin/calcineurin-nuclear factor of activated T cells (NFAT) pathway is a prototypical effector of SOCE (Srikanth and Gwack, 2013). To examine the putative role of NFAT in miR-184 induction, HPEK were incubated with the indirect calcineurin inhibitor cyclosporin A (CsA) prior to high Ca²⁺ challenge. As shown in FIG. 2, treatment with 1 μM CsA inhibited Ca²⁺-dependent induction of miR-184 by 70%. Together, these observations indicates that SOCE mediates the induction of miR-184 in HPEK and this occurs at least partly through the Ca²⁺⁻calmodulin/calcineurin-NFAT axis.

Effects of miR-184 on Keratinocyte Growth

The functional impact of miR-184 on HPEK behaviour was assessed using the MTT (3-(4,5-dimethylthiazol-2-yl)-2,5-diphenyltetrazolium bromide) reduction assay. Metabolic activity was reduced by around 40% in proliferating HPEK transfected with a miR-184 mimic compared control cells (FIG. 3a) although this was not significant. By comparison, live/dead cell staining with the Trypan Blue exclusion assay indicated a 17% drop in cell viability (FIG. 3b). Cell cycle analyses revealed that the proportion of HPEKs in G1 rose from 63.6% in controls to 72.7% in cells transfected with the miR-184 (FIG. 3c). A corresponding drop in the percentage of cells in G2 was also observed, from 13.7% in controls to 5.7% in miR-184. The proportion of HPEKs in S phase remained relatively unchanged. Hence, miR-184 appears to inhibit keratinocyte proliferation by slowing progression through the G1 phase of the cell cycle. These observations are consistent with the findings recently reported by Shalom-Feuerstein who showed that overexpression of miR-184 reduced keratinocyte proliferation in the basal layers of mouse epidermis (Nagosa et al., 2017).

Effects of miR-184 on Keratinocyte Differentiation

To uncover the effects of miR-184 on keratinocyte differentiation, IVL and cyclin E levels were examined in HPEK transfected with the miR-184 mimic Both proteins were barely detectable in proliferating HPEK maintained in low Ca²⁺ (FIG. 4a). In contrast, ectopic miR-184 triggered a 4-fold and 3-fold elevation in IVL and cyclin E expression, respectively (FIG. 4a,b). Conversely, both IVL and cyclin E were readily detected in HPEK cultured under high Ca²⁺ conditions. Transfection of a locked nucleic acid (LNA) miR-184 inhibitor lowered IVL and cyclin E expression by 60% and 90% respectively (FIG. 4c,d). Taken together, these observations implicate miR-184 in the regulation of the cyclin E pathway proposed by Gandarillas and colleagues in which cyclin E hyperactivation causes DNA damage that in turn signals for growth arrest and subsequent keratinocyte differentiation (Freije et al., 2012, Zanet et al., 2010).

Induction of DNA damage and p21 by miR-184

Given the observed miR-184-dependent induction of cyclin E (FIG. 4a,b) and the ability of cyclin E to promote DNA damage in HPEK (Freije et al., 2012, Zanet et al., 2010), it was hypothesised that ectopic miR-184 would trigger markers of DNA damage. Levels of γH2AX, a biomarker of DNA double-strand breaks, were elevated in HPEK transfected with miR-184 mimic compared to their counterparts transfected with a control mimic (FIG. 5a). Furthermore, cells transfected with miR-184 mimic had brighter/more γH2AX foci when visualised by fluorescence microscopy (FIG. 5b). Finally, we investigated the effect of miR-184 on the p21 CDK inhibitor, which previous studies showed was upregulated by cyclin E in HPEK (Freije et al., 2012). As shown in FIG. 5c,d exogenous miR-184 mimic increased p21 expression by 2.5-fold at the transcript level and 2-fold at the protein level. Taken together, our findings uncover a mechanism whereby SOCE triggers miR-184 during keratinocyte differentiation. In turn, miR-184 evokes DNA damage and p21 expression, the latter of which may explain the ability of miR-184 to slow keratinocyte progression through G1 phase of the cell cycle (FIG. 3c). Questions remain though, about the mechanisms by which accentuation of miR-184 promotes DNA damage. The model of Gandarillas and colleagues (Freije et al., 2012, Zanet et al., 2010) would suggest that elevation of cyclin E drives DNA damage. It is proposed that in addition targeting the Notch-dependent pathway (Nagosa et al., 2017), miR-184 serves to couple SOCE to the induction of cyclin E and DNA damage in order to elevate p21 and involucrin expression (FIG. 5e).

### EXAMPLE 2—MICRORNA-184 ACTIVATION IN WOUNDED KERATINOCYTES

As outlined in Example 1, it was observed that miR-184 was induced in human primary epidermal keratinocytes (HPEK) exposed to elevated extracellular Ca²⁺ but not other differentiation agents. The Ca²⁺-dependent induction of miR-184 was impaired when store-operated calcium entry (SOCE) was blocked with pharmacologic inhibitors or by silencing the expression of ORAI1, the predominant SOCE channel (Gudlur and Hogan, 2017). Given that keratinocyte migration from the basal layer to the stratum corneum is a fundamental element of epidermal differentiation, the inventors hypothesized that miR-184 may be induced during keratinocyte migration in wounded monolayers.

The inventors maintained the HPEK in low Ca²⁺ (0.07 mM) medium and allowed them to approach confluence in 6-well plates prior to wounding. Cells were harvested 5 days later and miR-184 expression assessed as described above. As shown in FIG. 6a, a 50-fold induction of miR-184 was observed in the scratched monolayers compared to their unscratched counterparts. Inhibition of SOCE with Gd³⁺ or BTP2 abolished the induction of miR-184 (FIG. 6b,c). As little or no miR-184 was detected in monolayer HPEK in low Ca²⁺ media (Roberts et al., 2013), these observations suggest that wounding sensitises HPEK to evoke de novo miR-184 expression under low Ca²⁺ conditions. In contrast, when extracellular Ca²⁺ was raised to 1.5 mM to 5 days to evoke HPEK differentiation, only a relatively modest 10-fold rise in miR-184 expression was detected (FIG. 6d). Given that high Ca²⁺ induces miR-184 expression (Richardson et al., 2018), this suggests that further upregulation of miR-184 in differentiating HPEK is blunted compared to the scale of the response observed in proliferating cells. Nevertheless, it appears that miR-184 induction is a response to wounding in both proliferating and differentiating keratinocytes.

The long non-coding RNA UCA1 (urothelial carcinoma associated 1) has been identified as a competing endogenous RNA (ceRNA) with four predicted miR-184-binding sites (Zhou et al., 2017). The expression of UCA1 in the HPEK was therefore examined. As shown in FIG. 6e, an 11-fold induction of UCA1 was observed in the scratched monolayers compared to their unscratched counterparts. Further, blockade of SOCE with Gd³⁺ or BTP2 reduced UCA1 induction by around 50% (FIG. 6f,g). Thus UCA1 appears to be co-induced with its miR-184 target in wounded HPEK and the upregulation of UCA1 was at least partly dependent on SOCE.

The direct impact of miR-184 on HPEK migration was then evaluated. The cells were loaded with a synthetic miR-184 mimic (GE Dharmacon) using nucleofection and monolayers scratched as they approached confluence. As shown in FIG. 7a, exogenous miR-184 led to a significant increase in the rate of HPEK migration compared to control cells loaded with a non-targeting oligonucleotide. In contrast, blockade of miR-184 activity using a locked nucleic acid (LNA) inhibitor (Qiagen) impaired HPEK migration (FIG. 7b). The linear regression of the 12-36 h period from the relative migration rates (FIG. 7c) yielded migration indices of 0.53±0.11 per hour for miR-184-loaded HPEK, compared to 0.15±0.05, 0.13±0.03 and 0.04±0.02 per hour for cells loaded with control mimic, control inhibitor and miR-184 inhibitor, respectively. In other words, elevation of miR-184 appeared to enhance keratinocyte migration 3-fold while miR-184 inhibition decelerated keratinocyte migration 3-fold.

Taken together, the results indicate that miR-184 is strongly upregulated in proliferating wounded keratinocyte monolayers and that SOCE mediates such elevation of miR-184. In addition, the lncRNA UCA1, which serves as a miR-184 “sponge” appears to be concomitantly raised with miR-184 and UCA1 induction also depends partly on SOCE. Exogenous miR-184 accelerated while anti-sense miR-184 inhibitor attenuated keratinocyte migration, suggesting miR-184 enhancement may be a useful approach to promote re-epithelisation during wound healing.

The forgoing embodiments are not intended to limit the scope of the protection afforded by the claims, but rather to describe examples of how the invention may be put into practice.

### REFERENCES

Amelio I, Lena A M, Viticchie G, Shalom-Feuerstein R, Terrinoni A, Dinsdale D, et al. miR-24 triggers epidermal differentiation by controlling actin adhesion and cell migration. J Cell Biol 2012; 199(2):347-63.
Bertero T, Gastaldi C, Bourget-Ponzio I, Imbert V, Loubat A, Selva E, et al. miR-483-3p controls proliferation in wounded epithelial cells. FASEB J 2011; 25(9):3092-105.
Bikle D D. Vitamin D regulated keratinocyte differentiation. J Cell Biochem 2004; 92(3):436-44.
Eckhart L, Lippens S, Tschachler E, Declercq W. Cell death by cornification. Biochim Biophys Acta 2013; 1833(12):3471-80.
Eichhorn S W, Guo H, McGeary S E, Rodriguez-Mias R A, Shin C, Baek D, et al. mRNA Destabilization Is the Dominant Effect of Mammalian MicroRNAs by the Time Substantial Repression Ensues. Mol Cell 2014; 56(1):104-15.
Eming S A, Martin P, Tomic-Canic M. Wound repair and regeneration: mechanisms, signaling, and translation. Science translational medicine 2014; 6(265):265sr6.
Freije A, Ceballos L, Coisy M, Barnes L, Rosa M, De Diego E, et al. Cyclin E drives human keratinocyte growth into differentiation. Oncogene 2012; 31(50):5180-92.
Gudlur A, Hogan P G. The STIM-Orai Pathway: Orai, the Pore-Forming Subunit of the CRAC Channel. Adv Exp Med Biol 2017; 993:39-57.
Guest J F, Ayoub N, Mcllwraith T, Uchegbu I, Gerrish A, Weidlich D, et al. Health economic burden that wounds impose on the National Health Service in the U K. BMJ Open 2015; 5(12):e009283.
Hildebrand J, Rutze M, Walz N, Gallinat S, Wenck H, Deppert W, et al. A comprehensive analysis of microRNA expression during human keratinocyte differentiation in vitro and in vivo. J Invest Dermatol 2011; 131(1):20-9.
Huntzinger E, Izaurralde E. Gene silencing by microRNAs: contributions of translational repression and mRNA decay. Nat Rev Genet 2011; 12(2):99-110.
Karlsson T, Vahlquist A, Torma H. Keratinocyte differentiation induced by calcium, phorbol ester or interferon-gamma elicits distinct changes in the retinoid signalling pathways. J Dermatol Sci 2010; 57(3):207-13.
Li D, Li X, Wang A, Meisgen F, Pivarcsi A, Sonkoly E, et al. MicroRNA-31 Promotes Skin Wound Healing by Enhancing Keratinocyte Proliferation and Migration. J Invest Dermatol 2015; 135(6):1676-85.
Li X, Li D, Wang A, Chu T, Lohcharoenkal W, Zheng X, et al. MicroRNA-132 with Therapeutic Potential in Chronic Wounds. J Invest Dermatol 2017; 137(12):2630-8.
Livak K J, Schmittgen T D. Analysis of relative gene expression data using real-time quantitative PCR and the 2(−Delta Delta C(T)) Method. Methods 2001; 25(4):402-8.
Nagosa S, Leesch F, Putin D, Bhattacharya S, Altshuler A, Senor L, et al. microRNA-184 Induces a Commitment Switch to Epidermal Differentiation. Stem cell reports 2017; 9(6):1991-2004.
Numaga-Tomita T, Putney J W. Role of STIM1- and Orail-mediated Ca2+ entry in Ca2+-induced epidermal keratinocyte differentiation. J Cell Sci 2013; 126(Pt 2):605-12.
Nunan R, Harding K G, Martin P. Clinical challenges of chronic wounds: searching for an optimal animal model to recapitulate their complexity. Disease models & mechanisms 2014; 7(11):1205-13.
Riemondy K, Hoefert J E, Yi R. Not miR-ly micromanagers: the functions and regulatory networks of microRNAs in mammalian skin. Wiley interdisciplinary reviews RNA 2014; 5(6):849-65.
Roberts J C, Warren R B, Griffiths C E, Ross K. Expression of microRNA-184 in keratinocytes represses argonaute 2. J Cell Physiol 2013; 228(12):2314-23.
Ross K, Whitaker M, Reynolds N J. Agonist-induced calcium entry correlates with STIM1 translocation. J Cell Physiol 2007; 211(3):569-76.
Ross K. Towards topical microRNA-directed therapy for epidermal disorders. J Control Release 2018; 269:136-47.
Sen C K, Gordillo G M, Roy S, Kirsner R, Lambert L, Hunt T K, et al. Human skin wounds: a major and snowballing threat to public health and the economy. Wound repair and regeneration 2009; 17(6):763-71.
Srikanth S, Gwack Y. Orail-NFAT signalling pathway triggered by T cell receptor stimulation. Molecules and cells 2013; 35(3):182-94.
Usui M L, Mansbridge J N, Carter W G, Fujita M, Olerud J E. Keratinocyte migration, proliferation, and differentiation in chronic ulcers from patients with diabetes and normal wounds. J Histochem Cytochem 2008; 56(7):687-96.
Vandenberghe M, Raphael M, Lehen'kyi V, Gordienko D, Hastie R, Oddos T, et al. ORAI1 calcium channel orchestrates skin homeostasis. Proc Natl Acad Sci USA 2013; Wang T, Feng Y, Sun H, Zhang L, Hao L, Shi C, et al. miR-21 regulates skin wound healing by targeting multiple aspects of the healing process. Am J Pathol 2012; 181(6):1911-20.
Wang T, Zhao N, Long S, Ge L, Wang A, Sun H, et al. Downregulation of miR-205 in migrating epithelial tongue facilitates skin wound re-epithelialization by derepressing ITGA5. Biochim Biophys Acta 2016; 1862(8):1443-52.
Whittam A J, Maan Z N, Duscher D, Wong V W, Barrera J A, Januszyk M, et al. Challenges and Opportunities in Drug Delivery for Wound Healing. Adv Wound Care (New Rochelle) 2016; 5(2):79-88.110(50):E4839-48.
Yang X, Wang J, Guo S L, Fan K J, Li J, Wang Y L, et al. miR-21 promotes keratinocyte migration and re-epithelialization during wound healing. Int J Biol Sci 2011; 7(5):685-90.
Yu J, Ryan D G, Getsios S, Oliveira-Fernandes M, Fatima A, Lavker R M. MicroRNA-184 antagonizes microRNA-205 to maintain SHIP2 levels in epithelia. Proc Natl Acad Sci USA 2008; 105(49):19300-5.
Yu J, Peng H, Ruan Q, Fatima A, Getsios S, Lavker R M. MicroRNA-205 promotes keratinocyte migration via the lipid phosphatase SHIP2. FASEB J 2010; 24(10):3950-9.
Zanet J, Freije A, Ruiz M, Coulon V, Sanz J R, Chiesa J, et al. A mitosis block links active cell cycle with human epidermal differentiation and results in endoreplication. PLoS One 2010; 5(12):e15701.
Zhou Y, Wang X, Zhang J, He A, Wang Y L, Han K, et al. Artesunate suppresses the viability and mobility of prostate cancer cells through UCA1, the sponge of miR-184. Oncotarget 2017; 8(11):18260-70.

1. A composition comprising a miR-184 modulator for regulating keratinocyte migration and/or differentiation.

2. The composition as claimed in claim 1, wherein the miR-184 modulator is adapted to increase expression or prevalence of miR-184 so as to promote keratinocyte migration and/or differentiation.

3. The composition as claimed in claim 2, wherein the promotion of keratinocyte migration and/or differentiation is in damaged or defective keratinocytes.

4. The composition as claimed in claim 3, wherein the promotion of keratinocyte differentiation is for re-epithelialization.

5. The composition as claimed in preceding claim, wherein the composition is for use as a medicament.

6. The composition as claimed in any preceding claim, wherein the composition is for use in the prevention, management, amelioration or treatment of damaged or defective keratinocytes in a skin condition or disease.

7. The composition as claimed in claim 6, wherein the skin condition comprises a wound.

8. The composition as claimed in claim 7, wherein the wound comprises a chronic wound.

9. The composition as claimed in any preceding claim, where the composition further comprises bioavailable calcium.

10. The composition as claimed in any preceding claim, wherein the composition further comprises a store-operated calcium entry modulator.

11. The composition as claimed in claim 10, wherein the store-operated calcium entry modulator comprises a calcium release-activated channel (CRAC) inhibitor.

12. The composition as claimed in claim 11, wherein the calcium release-activated channel (CRAC) inhibitor comprises BTP2 and/or derivatives thereof.

13. The composition as claimed in claim 1, wherein the miR-184 modulator for decreasing expression or prevalence of miR-184 so as to prevent or reduce keratinocyte migration and/or differentiation.

14. The composition as claimed in claim 13, wherein the prevention or reduction of keratinocyte migration and/or differentiation is in keratinocytes.

15. The composition as claimed in claim 14, wherein the prevention or reduction of keratinocyte differentiation is for the prevention or reduction of re-epithelialization.

16. The composition as claimed in any one of claims 13 to 15, wherein the composition is for use as a medicament.

17. The composition as claimed in any preceding claim, wherein the composition further comprises one or more pharmaceutically acceptable ingredients or excipients.

18. A composition comprising miR-184, or mimetic, or derivative thereof, for the promotion of keratinocyte migration and/or differentiation.

19. The composition as claimed in claim 18, wherein the promotion of keratinocyte migration and/or differentiation is in damaged or defective keratinocytes.

20. The composition as claimed in claim 19, wherein the promotion of keratinocyte differentiation is for re-epithelialization.

21. The composition as claimed in any one of claims 18 to 20, wherein the composition is for use as a medicament.

22. The composition as claimed in any one of claims 18 to 21, wherein the composition is for use in the prevention, management, amelioration or treatment of damaged or defective keratinocytes in a skin condition or disease.

23. The composition as claimed in claim 22, wherein the skin condition comprises a wound.

24. The composition as claimed in claim 23, wherein the wound comprises a chronic wound.

25. The composition as claimed in any one of claims 18 to 24, where the composition further comprises bioavailable calcium.

26. The composition as claimed in any one of claims 18 to 25, wherein the composition further comprises a store-operated calcium entry modulator.

27. The composition as claimed in claim 26, wherein the store-operated calcium entry modulator comprises a calcium release-activated channel (CRAC) inhibitor.

28. The composition as claimed in claim 27, wherein the calcium release-activated channel (CRAC) inhibitor comprises BTP2 and/or derivatives thereof.

30. The composition as claimed in any one of claims 18 to 28, wherein the composition further comprises one or more pharmaceutically acceptable ingredients or excipients.

31. A composition comprising an expression vector encoding miR-184, or a carrier molecule conjugated to miR-184, for the promotion of keratinocyte migration and/or differentiation.

32. The composition as claimed in claim 31, wherein the promotion of keratinocyte migration and/or differentiation is in damaged or defective keratinocytes.

33. The composition as claimed in claim 32, wherein the promotion of keratinocyte differentiation is for re-epithelialization.

34. The composition as claimed in any one of claims 31 to 33, wherein the composition is for use as a medicament.

35. The composition as claimed in any one of claims 31 to 34, wherein the composition is for use in the prevention, management, amelioration or treatment of damaged or defective keratinocytes in a skin condition or disease.

36. The composition as claimed in claim 35, wherein the skin condition comprises a wound.

37. The composition as claimed in claim 36, wherein the wound comprises a chronic wound.

38. The composition as claimed in any one of claims 31 to 37, where the composition further comprises bioavailable calcium.

39. The composition as claimed in any one of claims 31 to 27, wherein the composition further comprises a store-operated calcium entry modulator.

40. The composition as claimed in claim 39, wherein the store-operated calcium entry modulator comprises a calcium release-activated channel (CRAC) inhibitor.

41. The composition as claimed in claim 40, wherein the calcium release-activated channel (CRAC) inhibitor comprises BTP2 and/or derivatives thereof.

42. The composition as claimed in any one of claims 31 to 41, wherein the composition further comprises one or more pharmaceutically acceptable ingredients or excipients.
